(19) United States
(12) Patent Application Publication
(10) Pub. No.: US 2015/0230760 A1
(43) Pub. Date: Aug. 20, 2015

(54) METHODS, CIRCUITS, DEVICES, SYSTEMS AND COMPUTER EXECUTABLE CODE FOR OPERATING A MEDICAL DEVICE USING A HYBRID COMMUNICATION PATH

(71) Applicant: Q-CORE MEDICAL LTD., Netanya (IL)

(72) Inventor: Dennis Ian Schneider, Nashua, NH (US)

(73) Assignee: Q-CORE MEDICAL LTD., Netanya (IL)

(21) Appl. No.: 14/258,056

(22) Filed: Apr. 22, 2014

Related U.S. Application Data
(60) Provisional application No. 61/941,525, filed on Feb. 19, 2014.

(51) Int. Cl. A61B 5/00; A61M 5/172; A61M 5/142

(52) U.S. Cl. CPC A61B 5/7264; A61M 5/142; A61M 5/172; A61B 5/743; A61B 5/7475

(57) ABSTRACT

Disclosed is a medical device with a display and a therapeutic component to provide therapeutic functionality, and a controller to regulate operation of the therapeutic component and including processing logic to: (a) generate and render on the display an optical symbol indicative of an intended operational state of the therapeutic component; (b) receive from a user an operation code, and (c) responsive to validation of the operation code to enable the intended operational state.

### PRIORITY CLAIMS

The present application claims priority from U.S. Provisional Patent Application No. 61/941,525, titled: “Apparatus and Systems including Data Encoding with an Optical Medium and Methods for Carrying Out/Activating the Same”, filed by the inventor of the present application on Feb. 19, 2014, and is hereby incorporated by reference into the present application in its entirety.

### FIELD OF THE INVENTION

The present invention relates generally to the field of medical devices. More specifically, the present invention relates to methods, circuits, devices, systems and computer executable code for operating a medical device utilizing a hybrid communication path between the device and a management or authentication server.

### BACKGROUND

Medical devices operate for therapeutic and/or diagnostic uses. Some exemplary medical devices may be: blood pressure monitors which may monitor a patient's blood pressure and heart rate, electrical thermometers which may measure a patient's body temperature and many more.

Some medical devices may administer fluid to a patient via a conduit such as a flexible tube or a cassette including at least some flexible segments and some fixed segments. Some medical devices may monitor fluid flowing through its system and connected to one or more of a patient's bodily fluids. For example: an infusion pump which may be used to infuse fluids into a patient. In another example, a dialysis machine may pass a patient's blood through the machine to filter and get rid of toxins and excess fluids.

Some medical devices administering fluid or monitoring fluid may want to control the rate at which the fluid is flowing within the system.

A medical device may be used in a hospital, doctor or nurse's office or other medical treatment centers. Medical devices may also be used at patient's homes or personal environments.

### SUMMARY OF THE INVENTION

The present invention includes methods, circuits, devices, systems and computer executable code for operating a medical device using a hybrid communication path between the device and a management or authentication server. According to some embodiments of the present invention, a medical device may include: a display; a therapeutic component which may provide therapeutic functionality; and a controller which may regulate operation of the therapeutic component and including processing logic which may: (a) may generate and render on the display an optical symbol indicative of an intended operational state of the therapeutic component; (b) may receive from a user an operation code, and/or (c) responsive to validation of the operation code may enable the intended operational state. The medical device may further include a memory accessible by the controller; the memory may be configured to store medical device information. The medical device information may include at least one of the groups consisting of: therapeutic component operational log, medical device identification, software version information and medical device history. The optical symbol may further be indicative of medical device information retrievable from the memory. The intended operational state may be at least one of the states selected from the group consisting of: initialization, receive therapeutic operation parameters, begin therapeutic operation, end therapeutic information, log new medical device onto system, hold therapeutic functionality due to unusual event during operation and prepare medical device for repair. Optionally, the therapeutic component may be a fluid therapy pump. The medical device may further include a transceiver and the intended operational state may be indicative of a requested communication connection between the transceiver and a remote server, and the intended operational state be an open communication between the transmitter and the remote server so that the processing logic may be configured to receive secure information from the remote server through the transceiver.

According to some embodiments, a medical system may include: a medical device including a controller to generate and render on a display an optical symbol indicative of an intended operational state of the therapeutic component; and a first server to receive a processed optical symbol through a hybrid communication path, and to produce an operation code at least partially based on the processed optical symbol. Optionally, the medical device may be configured to receive the operation code and responsive to validation of the operation code may be configured to enable the intended operation state.

According to some embodiments, first server may be an authentication server and/or the first server may be a management server. The medical device may further include a display and a therapeutic component to provide therapeutic functionality. The controller may be further configured to regulate operation of the therapeutic component.

According to some embodiments, the system may include a medical device memory which may be accessible by the controller and may store medical device information. The optical symbol may be further indicative of at least a part of the medical device information stored in the memory. The first server may include a server memory which may include medical device information. The first server may be further configured to utilize both the processed optical symbol and the medical device information to produce the processed optical code. The medical device may include a transceiver, the intended operational state may be indicative of a requested communication connection between the transceiver and a remote server, and the intended operational state may be an open communication between the transmitter and the remote server so that the processing logic may be configured to receive secure information from the remote server through the transceiver. The remote server may be the first server. The medical device may include a near field transceiver, and the intended operational state may be indicative of a requested near field communication connection between the medical device and a proximate scanning device included in the hybrid communication path, and the intended operational state may be an open communication between the medical device and the scanning device.

According to some embodiments, the medical device may include a display; a diagnostic component to provide diagnostic functionality; and a controller to regulate operation of the diagnostic component and including processing logic to: (a) generate and render on the display an optical symbol indicative of a first state of the medical device; (b) receive from a user an operation code, and (c) responsive to validation of the operation code to enable transition to a second state of the medical device.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a functional block diagram of an exemplary medical system according to some embodiments of the present invention in which a hybrid communication path between a medical device and an management/authentication server may be utilized;

FIG. 2 is a functional block diagram including a hybrid communication path within a medical system including a scanning device and connectivity of the communication path to additional elements of the medical system;

FIGS. 3A-3C are functional block diagrams of a management/authentication server included in a medical system including connectivity of the authentication server to other elements of the medical system and peripheral elements of the management/authentication server;

FIG. 4 is a flowchart including the steps of an exemplary method by which medical system according to some embodiments of the present invention may perform authentication and/or management; and

FIGS. 5A-5K are flowcharts including the steps of an exemplary method by which medical system according to some embodiments of the present invention may perform authentication and/or management describing specific embodiments included within the spectrum of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Embodiments of the present invention may include apparatuses for performing the operations herein. This apparatus may be specially constructed for the desired purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs) electrically programmable read-only memories (EPROMs), electrically erasable and programmable read only memories (EEPROMs), magnetic or optical cards, or any other type of media suitable for storing electronic instructions, and capable of being coupled to a computer system bus.

The processes and displays presented herein are not inherently related to any particular computer or other apparatus. Various general purpose systems may be used with programs in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

According to some embodiments of the present invention, a medical device may include: a display; a therapeutic component which may provide therapeutic functionality; and

a controller which may regulate operation of the therapeutic component and including processing logic which may: (a) may generate and render on the display an optical symbol indicative of an intended operational state of the therapeutic component; (b) may receive from a user an operation code, and/or (c) responsive to validation of the operation code may enable the intended operational state. The medical device may further include a memory accessible by the controller; the memory may be configured to store medical device information. The medical device information may include at least one of the groups consisting of: therapeutic component operational log, medical device identification, software version information and medical device history. The optical symbol may further be indicative of medical device information retrievable from the memory. The intended operational state may be at least one of the states selected from the group consisting of: initialization, receive therapeutic operation parameters, begin therapeutic operation, end therapeutic information, log new medical device onto system, hold therapeutic functionality due to unusual event during operation and prepare medical device for repair. Optionally, the therapeutic component may be a fluid therapy pump. The medical device may further include a transceiver and the intended operational state may be indicative of a requested communication connection between the transceiver and a remote server, and the intended operational state be an open communication between the transmitter and the remote server so that the processing logic may be configured to receive secure information from the remote server through the transceiver.

According to some embodiments, a medical system may include: a medical device including a controller to generate and render on a display an optical symbol indicative of an intended operational state of the therapeutic component; and a first server to receive a processed optical symbol through a hybrid communication path, and to produce an operation code at least partially based on the processed optical symbol. Optionally, the medical device may be configured to receive the operation code and responsive to validation of the operation code may be configured to enable the intended operation state.

According to some embodiments, first server may be an authentication server and/or the first server may be a management server. The medical device may further include a display and a therapeutic component to provide therapeutic functionality. The controller may be further configured to regulate operation of the therapeutic component.

According to some embodiments, the system may include a medical device memory which may be accessible by the controller and may store medical device information. The optical symbol may be further indicative of at least a part of the medical device information stored in the memory. The first server may include a server memory which may include medical device information. The first server may be further configured to utilize both the processed optical symbol and the medical device information to produce the processed optical code. The medical device may include a transceiver, the intended operational state may be indicative of a requested communication connection between the transceiver and a remote server, and the intended operational state may be an open communication between the transmitter and the remote server so that the processing logic may be configured to receive secure information from the remote server through the transceiver. The remote server may be the first server. The medical device may include a near field transceiver, and the intended operational state may be indicative of a requested near field communication connection between the medical device and a proximate scanning device included in the hybrid communication path, and the intended operational state may be an open communication between the medical device and the scanning device.

According to some embodiments, the medical device may include a display; a diagnostic component to provide diagnostic functionality; and a controller to regulate operation of the diagnostic component and including processing logic to: (a) generate and render on the display an optical symbol indicative of a first state of the medical device; (b) receive from a user an operation code, and (c) responsive to validation of the operation code to enable transition to a second state of the medical device.

The present invention includes methods, circuits, devices, systems and computer executable code for operating a medical device using a hybrid communication path between the device and a management or authentication server. According to embodiments, a medical device to be activated or reconfigured may output an optical code/symbol. An operator of the device may scan the optical code/symbol using a networked optical scanning device such as a smart phone, QRcode scanner, barcode scanner, camera, cellular phone, touch screen computer, handheld device and more and convey the scanned information to a management or authentication server over a wireless or wired data network. The authentication server may compare the received information against a database containing records for the device or patient for which the device is intended and may assess appropriateness of the received encoded information. A message, which may include an operations code, responsive to the assessment may be generated by the authentication server and transmitted back to the scanning device or to a device functionally associated with the scanning device. A user of the scanning device may receive and input the authentication server message into the medical device, which medical device may use the message to: (1) set an operational configuration, (2) validate a current operational configuration, (3) confirm that the operational history allows further use of the medical device, (4) log a new medical device onto a system or fleet, (5) alarm/notify/flag medical devices needing maintenance, (6) enable an open communication path between the medical device and a remote server, (7) enable an open communication path between the medical device and a near field communication device and more.

A hybrid communication path used within a medical system may both enable increased safety and compliance with industry, governmental and regulatory safety requirements. A hybrid communication path may include two or more steps, where at least one of the steps requires user facilitation in order to be carried out. For example, for patient safety a medical device may not be completely remotely activate-able, a hybrid communication path may be utilized so that additional activation/management steps may be carried out substantially remotely with a user-involved in at least one of the steps for heightened security. Furthermore, the hybrid communication path may enable an efficient way to increase safety in the home environment, for example, by adding a user-involved confirmation step after setting a medical device parameters or otherwise.

While the term management/authentication server is used, it is understood, throughout this application that a single M/A server may carry out either management or authentication or both. Furthermore, as well known in the art, where one M/A server is described, a group of M/A servers may be used in conjunction to carry/out facilitate the described functionality.

It is understood that while a scanning device is described having both scanning and transceiving functionality, a singular or multiple configuration may be used so that a single device may carry out the functionality or two or more devices may be used to carry out the functionality. For example, a bar code and a smartphone may be used (multiple devices) or a smartphone may be used alone (singular device).

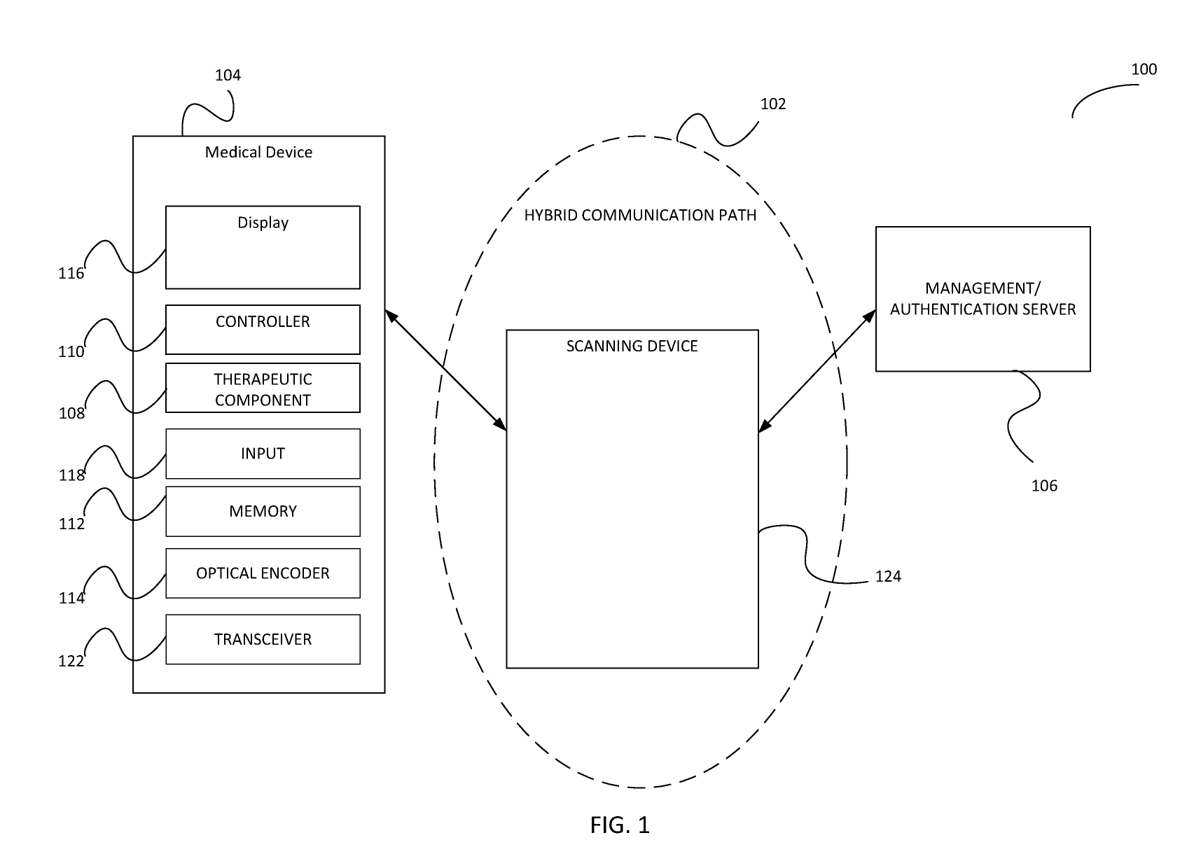

Turning to FIG. 1, depicted is a functional block diagram (100) of an exemplary medical system according to some embodiments of the present invention in which a hybrid communication path 102 between a medical device 104 and an M/A server 106 may be utilized. In a first state and before transitioning into a second state, medical device 102 may be configured to output an optical code via Hybrid communication path 102 to M/A server 106. M/A server 106 may decode and compare the received information against a database and assess appropriateness of the received information and relay a message back to medical device 104 via hybrid communication path 102. Depending on the message received from M/A server 106, medical device 104 may enter/transition/enable a second state or a third state (and more); depending on the outcome of the appropriateness of the received information and/or medical device 104 may be disabled.

According to some embodiments medical device 104 may include an infusion pump, peristaltic pump, syringe pump, fluid therapy pump, heart-lung machine, dialysis machine Sphygmomanometer, insulin pump, spirometer and more. Medical device 102 may have a therapeutic functionality such as: to pump fluid and/or medication intravenously to a patient, to filter the blood stream of a patient, to measure a patient's blood pressure, to evaluate/determine how well a patient is breathing and more. Medical device 104 may include one or more therapeutic components such as therapeutic component 108, to at least partially carry out the therapeutic functionality, for example a peristaltic pump may have a peristaltic mechanism to cause fluid to flow through an intravenous set to a patient; a dialysis machine may have a filter to filter out elements of a blood stream and a pump to cause a patient's blood to flow through the system, a Sphygmomanometer may have a pump to cause pressure on a patient's body (arm or otherwise), a spirometer may evaluate/determine the volume of air inspired and/or expired by a patient's lungs and more.

According to some embodiments, medical device 104 may include at least one controller/computer/processing logic such as controller 110, which may be configured to operate/control the medical device for example: controlling therapeutic modes, ensuring safety, accessing memory, activating Wifi and controlling the ancillary circuits of medical device 104.

According to some embodiments, medical device 104 may include one or more memories and/or buffer(s) to store information such as memory 112. Examples of information/data that may be stored within memory 112 include: an operational log of the medical device, medical device identification, error/alarm log records, treatment records, parameters entered by a user, drug library and more. The memory may be a separate block or may be embedded within the computer.

According to some embodiments, medical device 104 may include an optical encoder such as optical encoder 114 which may receive information from the memory and/or directly from controller 110 and produce/calculate an optical code. Optical encoder 114 may be embedded or an integral a part of controller 110 or may be a separate circuit. The optical code, produced by the optical encoder and/or the controller may be any type of image or symbol for example: a barcode, QR code, Qcode, linear barcode, picture, hologram, 2D or 3D image or a combination of these or otherwise and more. Data that may be encoded within the Optical Code may include medical device data, therapy related data, where/how to access M/A server, user information and generally any type of information accessible from controller 110 and/or memory 112 relating to medical device 110 and it's functionality and connectivity including the internal blocks of medical device 104 such as memory 112, therapeutic component 108 and any other block, confirmation code information and more. Furthermore, the information embedded in the optical code may be encoded in a multi-level encoding/encryption configuration so that some of the information is decodable/de-cryptable by a user scanning device and other information may be decodable/de-cryptable by an M/A server or otherwise. The multi-level encoding configuration may enable one or more of the levels to be encoded in a sound/stream method so that information is encoded into a sound and/or a stream of sounds.

According to some embodiments, medical device 104 may include a display such as display 116 which may include an Optical-Code-Displayable medium such as a screen, hologram emitter, keyboard (electronic or virtual), keys, buttons, switches, and more. The different inputs may be identical (such as two different touchscreens) or different mediums (such as a keyboard and a touchscreen). The input and the display may be two different mediums or may be at least functionally overlapping (for example a touchscreen).

According to some embodiments, medical device 104 may include a transceiver 122 configured to enable connectivity and data transmission and reception via a wireless network such as WIFI, cellular or otherwise. Transceiver 122 may include one or more local antenna and may be at least partially controlled by controller 110.

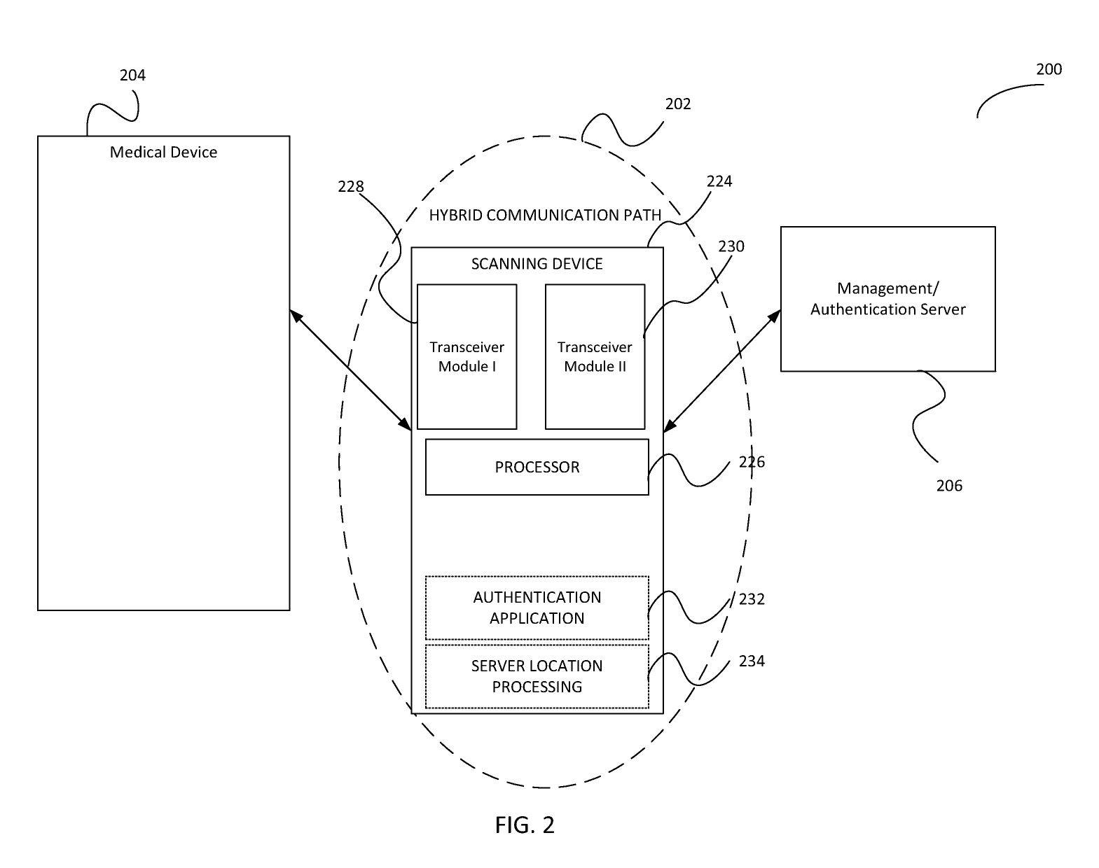

Turning now to FIG. 2, depicted is a functional block diagram 200 including a hybrid communication path 202 within a medical system including a scanning device 124 and connectivity of the communication path to other elements of the system. It is understood that medical device 204 and M/A server 206 are substantially similar to medical device 104 and M/A server 106 (respectively) of FIG. 1. Furthermore, hybrid communication path 202 may be substantially similar to hybrid communication path 102 of FIG. 1.

According to some embodiments, an optical code may be relayed from medical device 204 to M/A server 206 through hybrid communication path 202. Hybrid communication path 202 may be at least partially operated/controlled by a user. Some examples of a user include: an at home patient, an at home healthcare provider, a technician, a caregiver, a nurse, a doctor, a patient and more. The user may be in a hospital or other medical facility or may be at a non-medical environment such as a patient's home.

According to some embodiments, the user may activate or use an input peripheral to the M/A server such as scanning device 224. Scanning device 224 may be a cellphone, smart phone, camera, digital camera, computer, bar code scanner, QR code scanner and more. Scanning device 224 may be configured to receive or obtain the optical code by a first medium/method (such as a camera, CCD, scanner and more). The first medium may be user selected. Scanning device 224 may include a processor 226 to at least partially transform some of the information and/or the optical code. Processor 226 may at least partially decode at least one level of the optical code which may then be transformed and/or re-encoded and/or additional information may be added to the optical code resulting in a transformed/processed optical code. Furthermore, the additional information may be embedded within, at the beginning or end of the information decoded from the optical code information or added “on top” of that information.

According to some embodiments, the scanning device may encode additional information into the transformed optical code which may include location information provided by the scanning device, for example, if the scanning device includes a GPS or if the location can be deciphered for example by information associated with Wi-Fi antennas, cellular antennas and more.

Optionally, the scanning device may substantially maintain the optical code in its originally obtained form so that the optical code and the transformed optical code are substantially the same.

According to some embodiments, scanning device 224 may include a first transceiver such as transceiver module 228 which may relay the transformed optical code to M/A server 206 via a wireless or wired medium such as cellular, internet, Wi-Fi, Bluetooth, infrared, and more and may be user selected. Scanning device 224 may include a second transceiver such as transceiver module 230 to receive a server message from M/A server 206. Transceiver module 230 may receive and transmit information may relay/receive information using the same mediums discussed with regard to transceiver module 228. Transceiver module 228 and transceiver module 230 may transmit/receive via identical or different mediums. Optionally, transceiver module 228 and transceiver module 230 may be joined so that only one transceiving module may be needed.

According to some embodiments, scanning device 224 may include authentication application 232 which may be a dedicated application to supply or add information associated with the optical code and/or encoding of the optical code and/or may include M/A server 206 address or location and access information. Optionally, scanning device may include server location processing 234 to aid in accessing M/A server 206 based on address associated information received from the optical code and/or access data stored in scanning device 224. Optionally, M/A server 206 may be accessed at least partially using user inserted information such as a web address, phone number or otherwise.

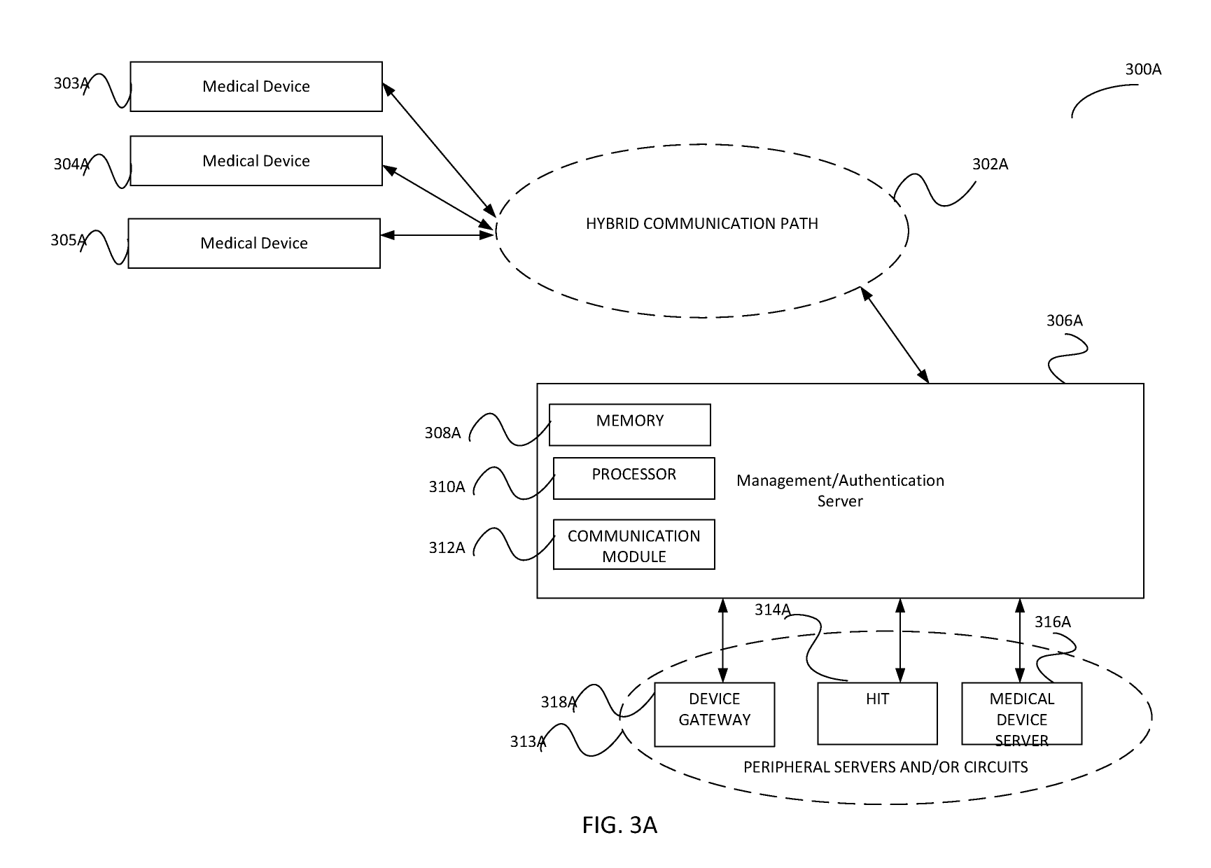

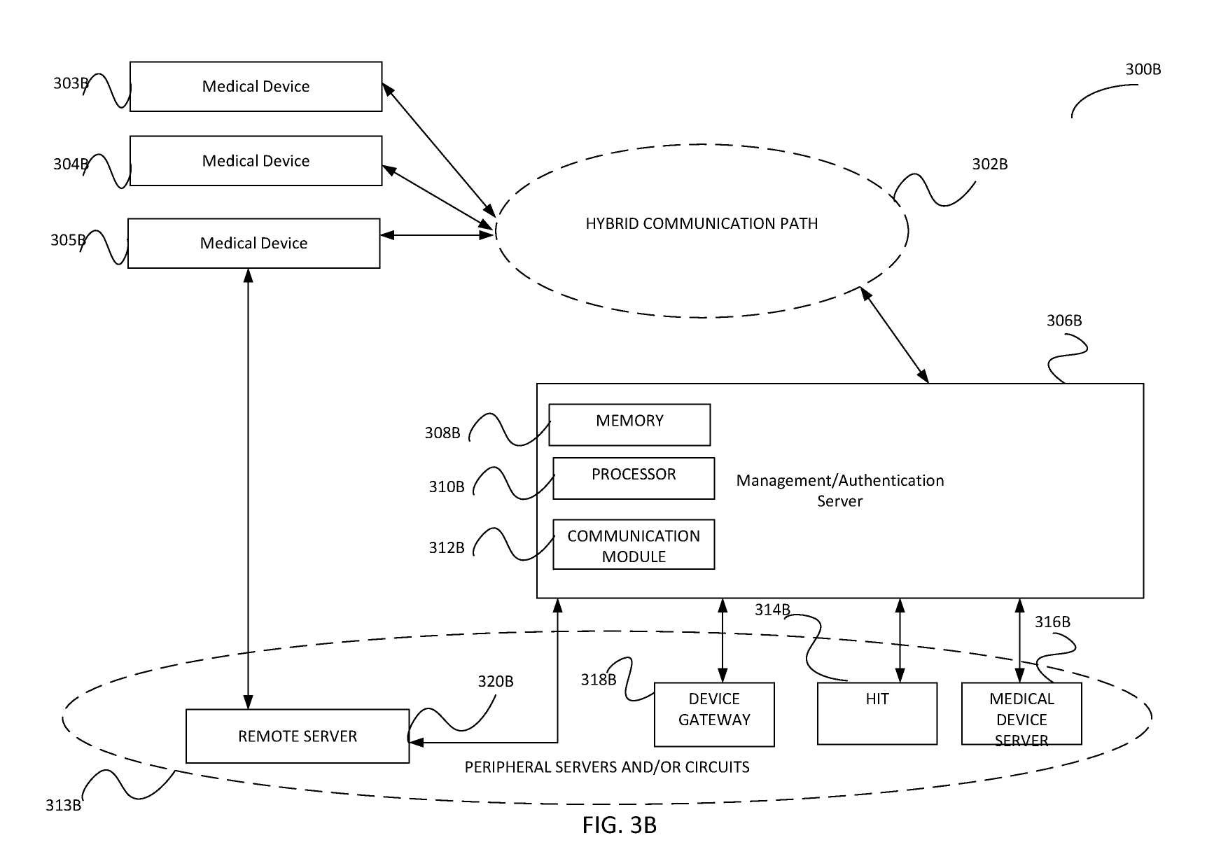

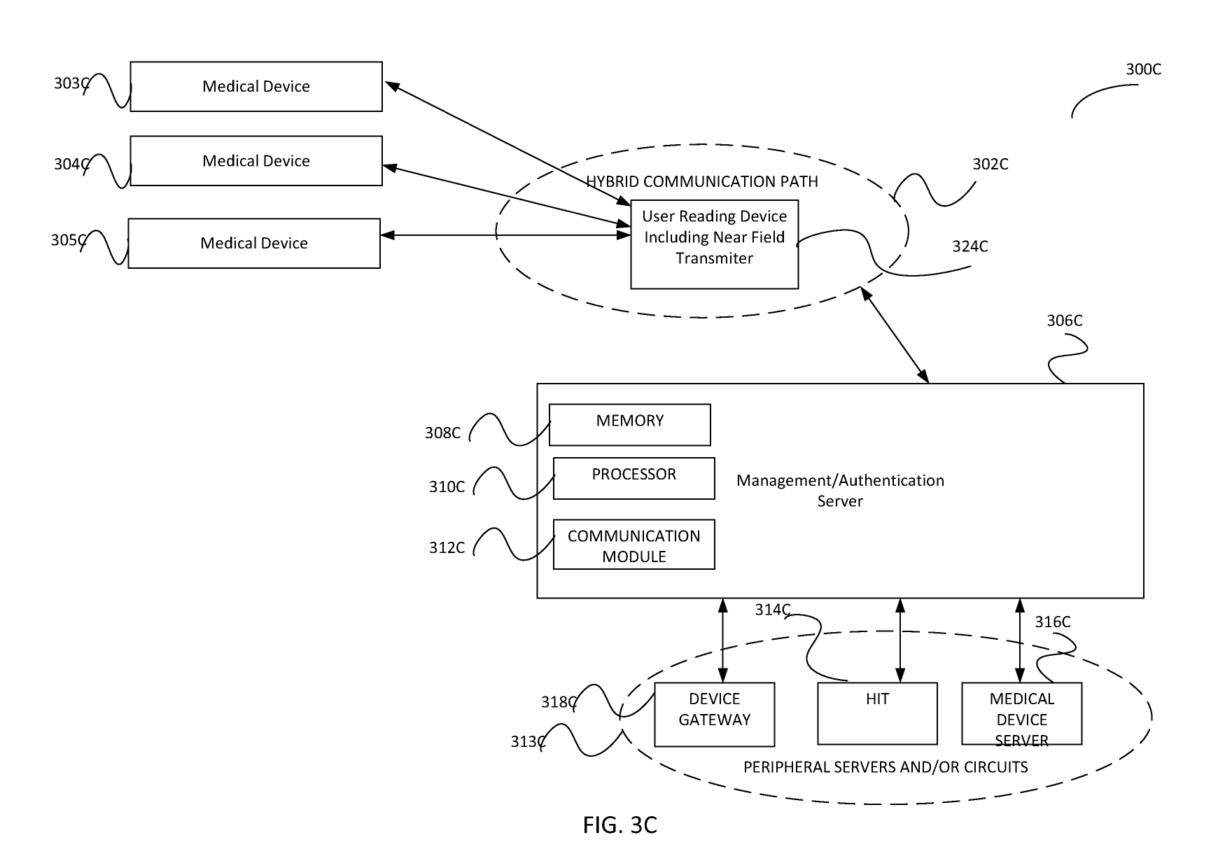

Turning to FIG. 3A, depicted is a functional block diagram of an M/A server 306A included in a medical system 300A including connectivity of M/A server 306A to other elements of the medical system 300A and peripheral servers and/or circuits (peripherals) 313A. It is understood that medical device 303A, 304A and 305A are each substantially similar to medical device 104 and that M/A server 306A may be substantially similar to M/A server 106 of FIG. 1. Furthermore, hybrid communication path 202 may be substantially similar to hybrid communication paths 102 of FIG. 1.

According to some embodiments, M/A server 306A may receive a transformed optical code from/via hybrid communication path 302A. M/A server 306A may include memory 308A to store information such as expected/base/comparative information. Some examples of information stored in memory 308A may include: a list of authorized users, expected medication for patient, EPR-electronic patient record, drug library, medical device information such as error codes and tables associated with proper or improper functioning of the device, expected software version for each medical device, minimal-battery-life as a function of the expected treatment, list of medical devices in service within a fleet of medical devices, list of stolen or missing medical devices within a fleet of medical devices, local parameters/definitions and more. M/A server 306A may include processor 310A to assess appropriateness of the received encoded information. Processor 310A may compare/calculate/process and assess the appropriateness based on the received processed optical code as well as additional information either stored on memory 308 or accessible through/at peripherals 313A. M/A server 306A may also store/update information stored in memory 308A and/or peripherals 313A based on the processing result and the specific embodiment. Note that processor 310A may also decode the received optical code and/or one or more layers of the received optical code. M/A server 306A may also include communication module 312A to receive the processed code as well as to access peripherals and communicate with them 313A.

According to some embodiments, peripherals 313A may include hospital information technology (HIT) server 314A, medical device server 316A and/or local device gateway server 318A and more. HIT server 314A may include information regarding billing, pharmacy, drug libraries and electronic patient records and more. Device gateway server 318A may include maintenance and/or management information and control regarding one or more associated medical devices or a fleet of medical devices (such as medical devices 303A-305A), Medical device server 316A may include software versions, information regarding operation errors, maintenance information and more.

According to some embodiments, M/A server 306A may return a server message based on analyzing/processing of the received information and, optionally, may also utilize/use data stored on peripherals 313A or accessible via peripherals 313A. M/A server 306A may return a server message based on a confirmation of storing updated/new data on memory 308A and/or peripherals 313A. The server message may include instructions to a user and/or operational code, these may either instruct a user how to proceed, may cause a message to be presented on a medical device, may cause a message to be presented on the scanning device and/or may enable/disable a medical device to transition into a second mode and/or enable transition into an intended mode and more.

According to some embodiments, the server message may be encoded in many methods and may also be in a multi-level configuration so that some of the data is encrypted and may be decipherable by the user-device. The server message may be encoded in a binary, optical or sound method so that the data is encoded into a sound/stream of sounds.

Turning to FIG. 3B depicted is a medical system 300B which is understood to be substantially similar to medical system 300A. Elements/blocks 302B-318B are substantially similar to elements 302A-318A (accordingly). Medical system further includes a remote server such as remote server 320B. It is understood that remote server 320B may be an additional server or may overlap at least partially in functionality or circuitry with servers 306B, 314B, 316B and 318B. Medical system 300B may be configured so that server 320B and/or medical device 305A (for example) may send a communication connection request and in response medical device 305A may display an optical code which may include medical device identification information. Similarly to as described above, medical device 305B may be confirmed/authenticated by M/A server 306B via hybrid communication path 302B. Upon receipt of the confirmation code from the M/A server the medical device may enable secure/safe communication between remote server 320B and medical device 305B. Server 320B and medical device 305B may communicate directly or via M/A server 306B or otherwise. Secure/safe/open communication between a medical device and/or server may (such as medical device 305B and remote server 320B) may include transfer of information that may require a heightened security because it should only be seen by authorized users or because the information may cause an update of the medical device that requires a higher level of security for example for regulatory or health safety reasons. Secure information may include: patient related information, drug library information, medical device configuration information and more.

According to some embodiments, the secure information may cause/enable the medical device to update a secured operational configuration such as: auto remote programming of a treatment to medical device 305B, update of software stored on medical device 305B, update of drug library stored on medical device 305B, receive of a dose programming for medical device 305B for administering drugs to a patient, auto documentation of medical device 305B operating status and more.

According to some embodiments, system 300B may further enable, diagnosis of connectivity issues if medical device 305B is failing to connect directly to a remote server 320B, for example by receiving information to M/A server 306B from medical device 305 via scanning device included in hybrid communication path 302A such as are the medical device transmitters operating properly and more and analyzing what may be causing the failure.

Turning to FIG. 3C depicted is a medical system 300C which is understood to be substantially similar to medical system 300A. Elements/blocks 302C-318C are substantially similar to elements 302A-318A (accordingly). Scanning device 324C of hybrid communication path 302C includes a near field transceiver such as a Bluetooth, infrared or otherwise. Medical system 300C may be configured so that scanning device 324C and/or medical device 305C (for example) may send a near field communication connection request and in response medical device 305C may display an optical code which may include medical device identification information. Similarly to as described above, medical device 305C may be confirmed/authenticated by M/A server 306C via hybrid communication path 302C. Upon receipt of the confirmation code from the M/A server the medical device may enable secure/safe near field communication between scanning device 324C and medical device 305C.

Returning to FIG. 1, a server message may be relayed to a user at the hybrid communication path. Upon receipt of the server message a user may receive instructions how to proceed and/or may receive an operational code. The user may relay the operational code, received from server 106 to scanning device 124 or otherwise to a user. The user may relay the code and/or message to medical device 104 (for example via input 118), which may cause the medical device to transition into a second or third mode, enable an intended mode or may disable the medical device or otherwise, bases on the input message or operational code and may further display a message associated with the received code.

According to some embodiments, the optical symbol may include encoded confirmation code information such as: pseudo-random code, changing/updated information, time dependent information and more. M/A server 106 may utilize the confirmation code information to produce the operation code so that it is temporary, time dependent, case dependent and/or changing and/or confirmation-code-dependent. Accordingly, the operation code may only be valid for a given time and the received operation code may be variable/unfixed. An unfixed and/or variable operation code may cause an operation code to be variable even if a user inputs that same parameters daily and/or the same variables are used on an identical medical device and may also cause an operation code to become invalid if not used within a predetermined/predefined time. Note that medical device 104 may store information to memory 112 so that controller 110 may confirm/analyze the received operational code and determine/confirm the next step to be taken by medical device 104. The stored information may include the confirmation code information and a table correlating medical device responses to received operational code and/or an algorithm for determining the medical device response and more.

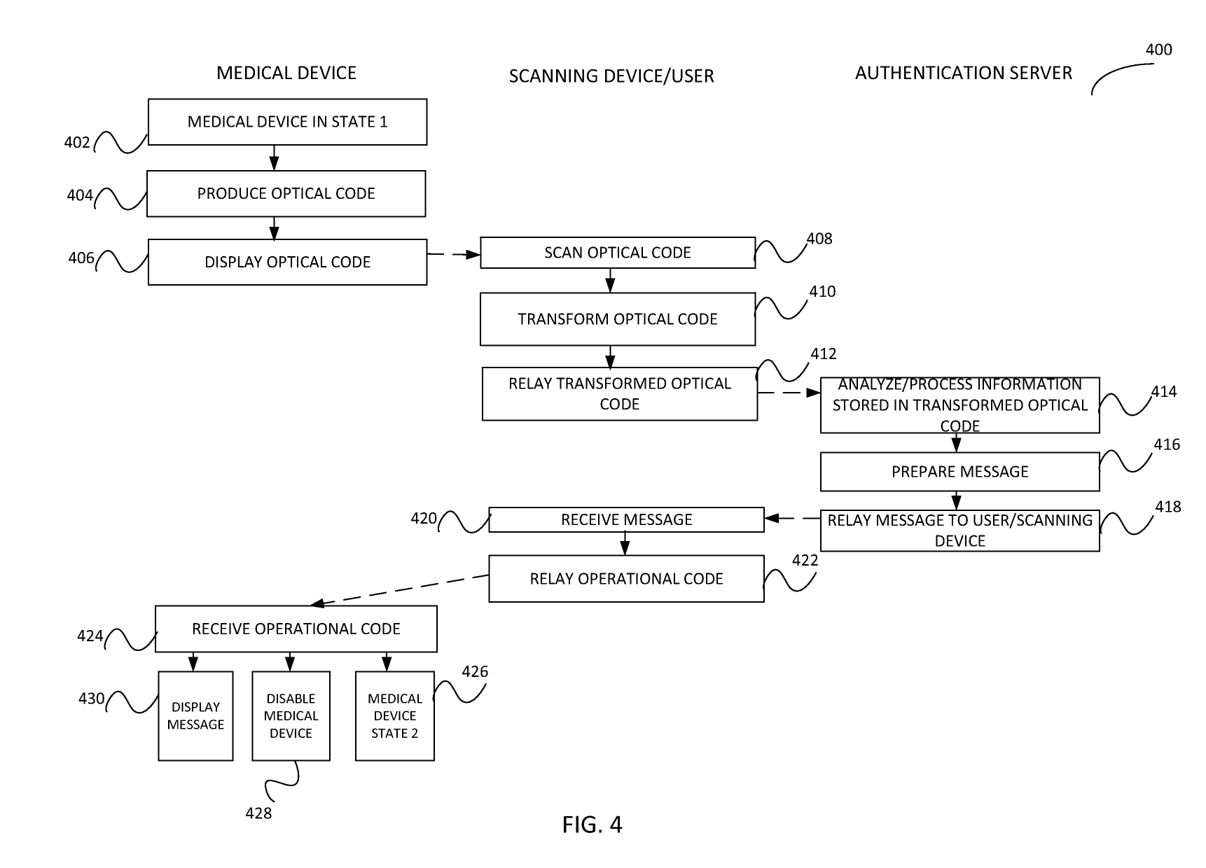

Turning to FIG. 4, shown is a flowchart 400 including the steps of an exemplary method by which a medical system according to some embodiments of the present invention may perform authentication and/or management so that a medical device may safely transition from a first state to a second state utilizing an authentication or management step/process. A medical device may be in a first state or identify an intended state (step 402) and may automatically or based on a user request calculate or determine an optical code based on information stored in the medical device (step 404) and cause the optical code to be displayed (step 406). A scanning device, optionally operated by a user, may then scan/obtain the optical code from the medical device (step 408). Optionally, the scanning device may also transform the optical code (step 410). Transforming the optical code may include decoding, re-encoding in a new method, adding additional information, preparing the information for transition and more so that the transformed optical code may be a binary-type code, sound-based code, a new optical code or identical to the original optical code or otherwise. The scanning device may relay/transmit the transformed optical code to an M/A server (step 412). The M/A server may receive the transformed optical code and may analyze/process information stored in the transformed optical code as well as the M/A server and data accessible to the M/A server (step 414) and may prepare a message based comparing/analyzing the aforementioned information (step 416) and relay the message back to a user and/or scanning device and/or additional device associated with the user (step 418). The scanning device may receive the message which may include an operational code (step 420). A user may relay the operational code to the medical device (step 422). The medical device may receive the operational code (step 424) and that may cause the medical device to transition into a second state or enable the intended state (step 426), disable the medical device (step 428) and/or a message may be emitted on the display in response to the operational code (step 430) which may include further instructions for a user, for example: how to proceed, status of the medical device and more.

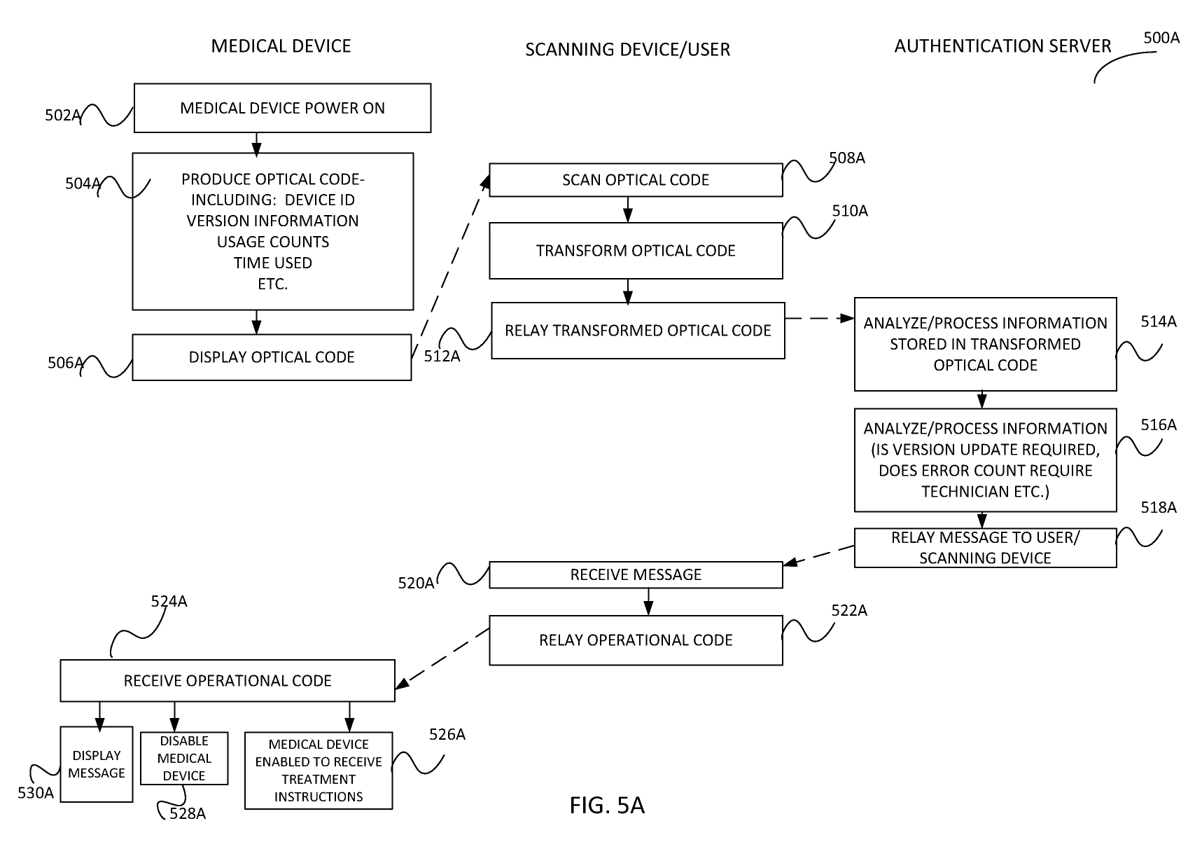

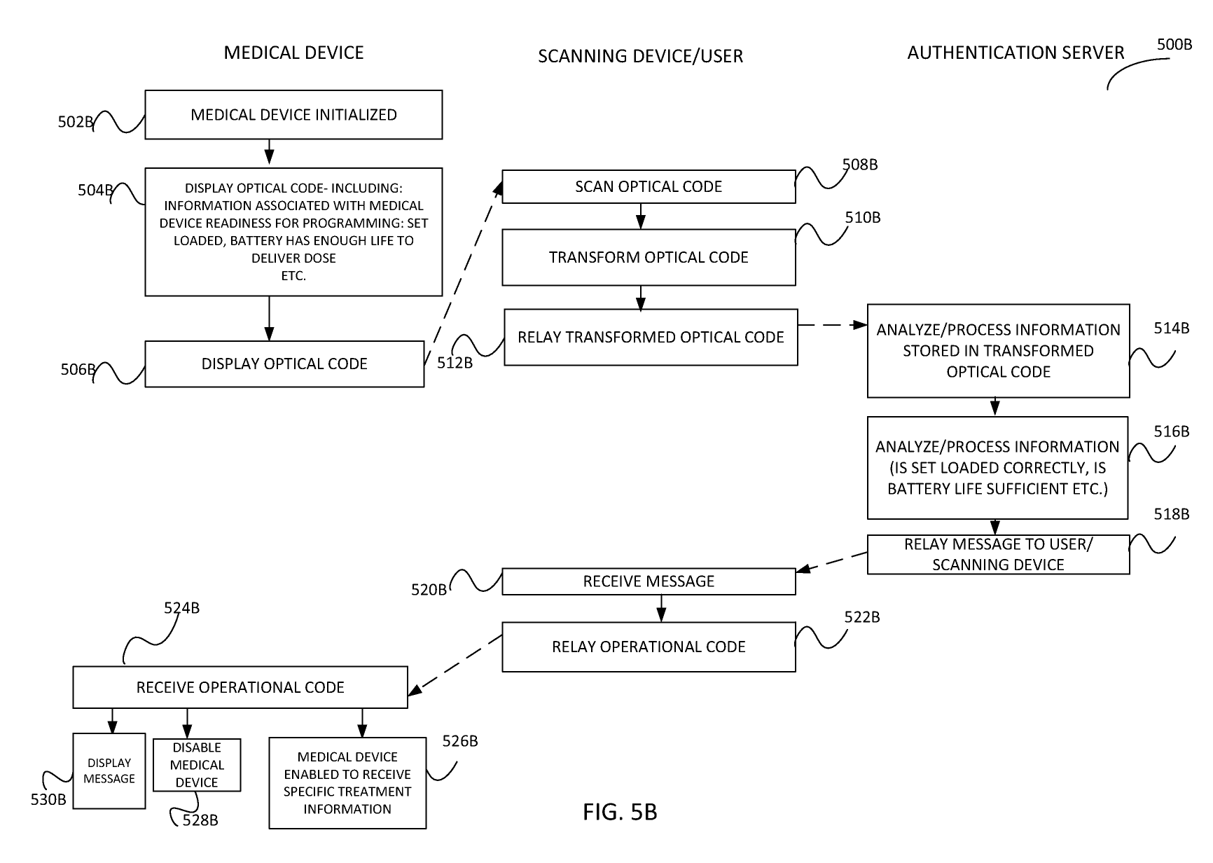

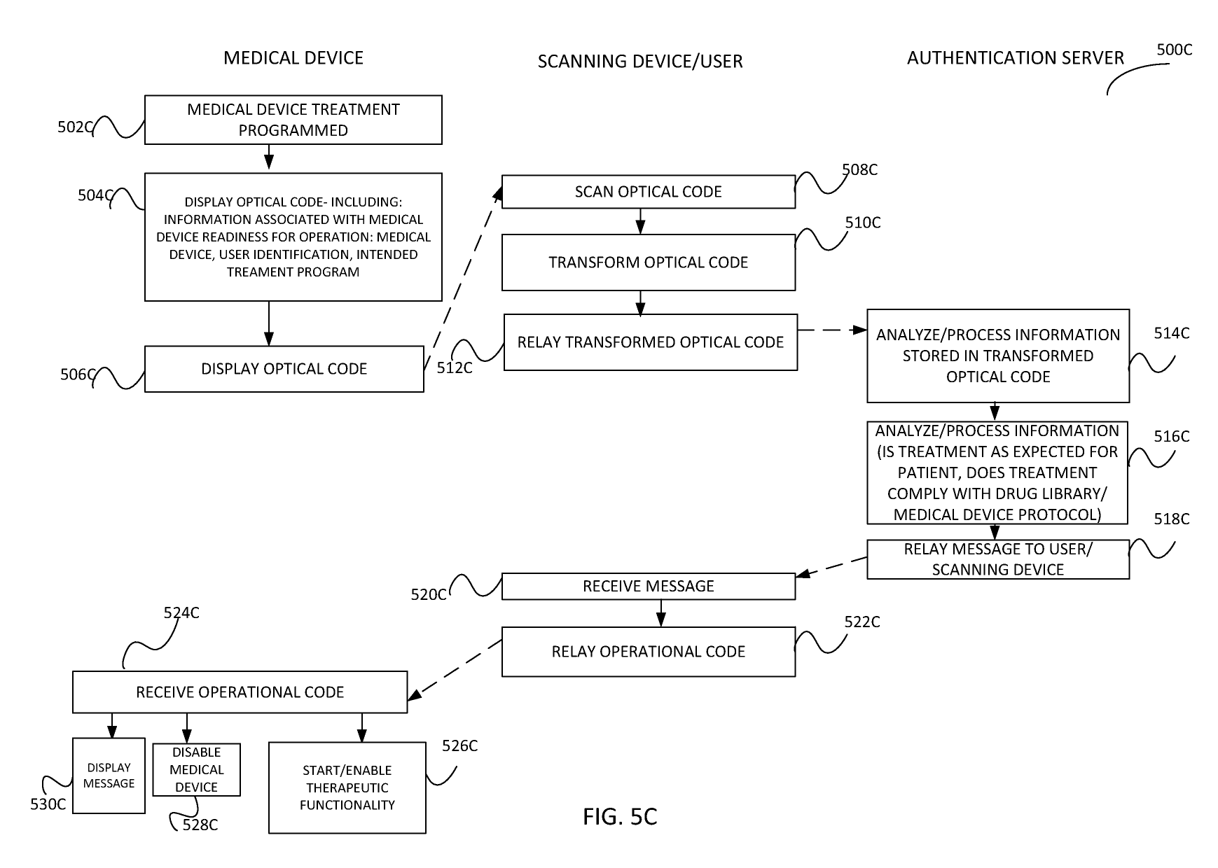

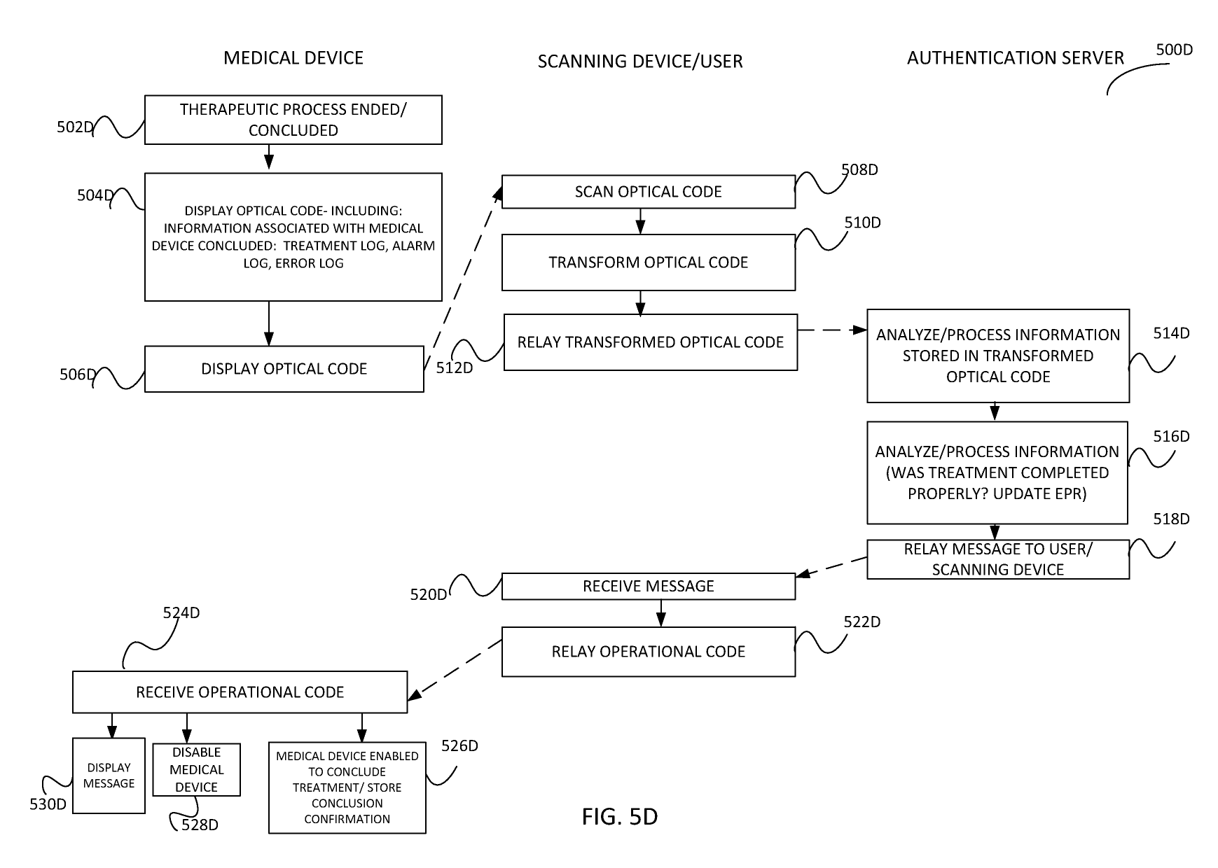

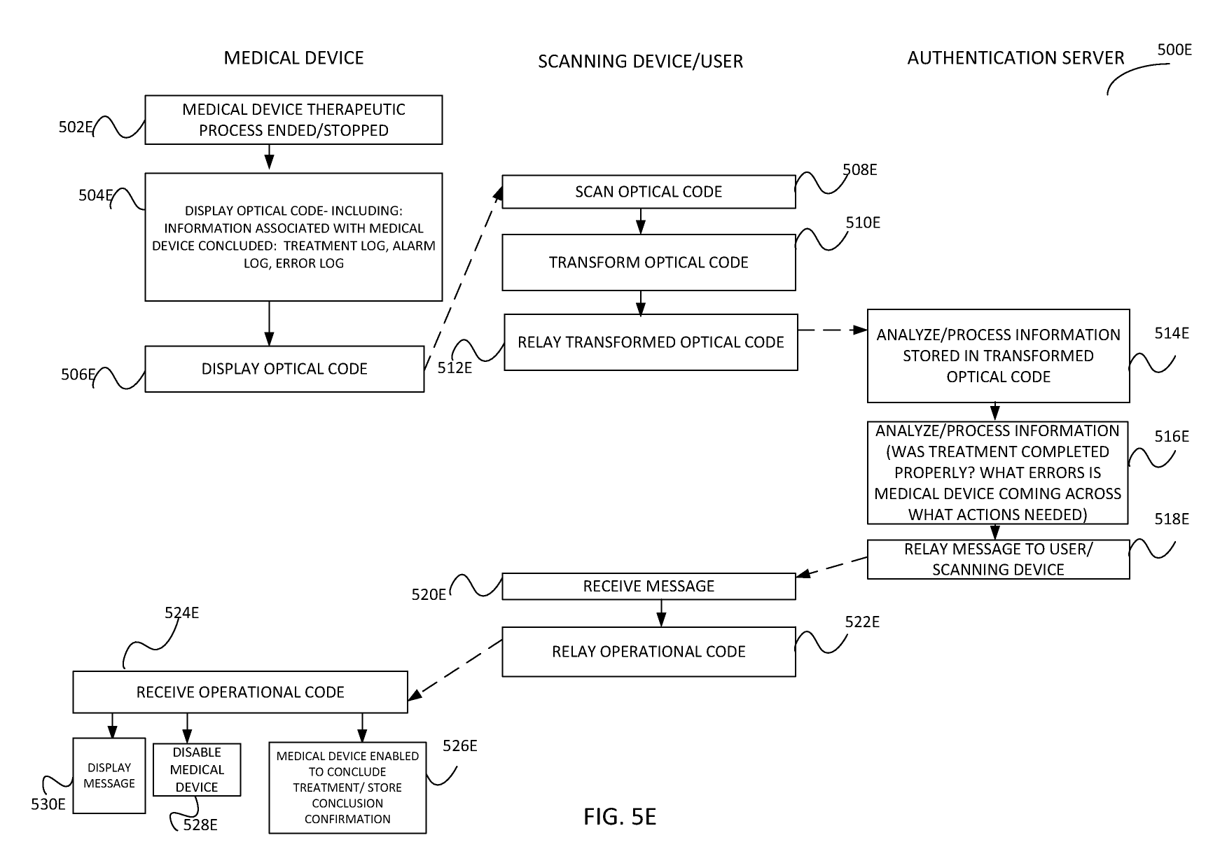

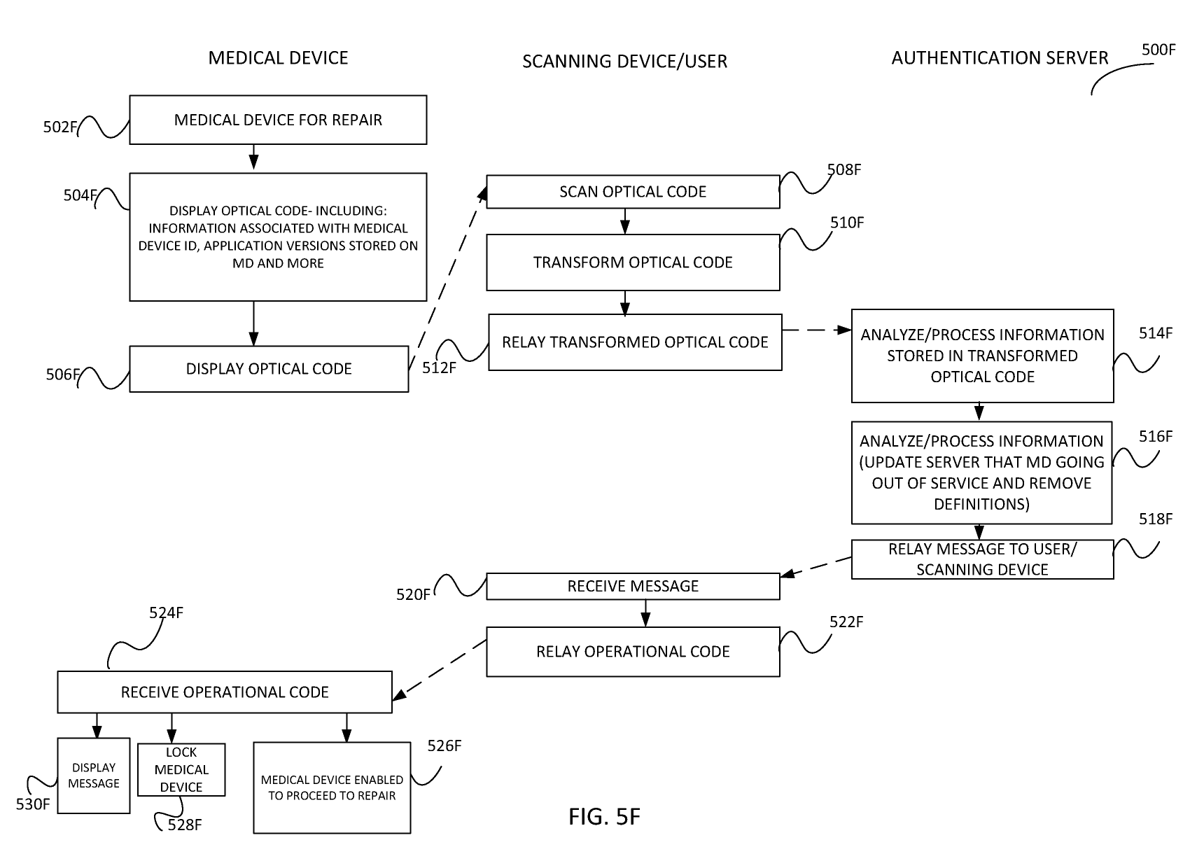

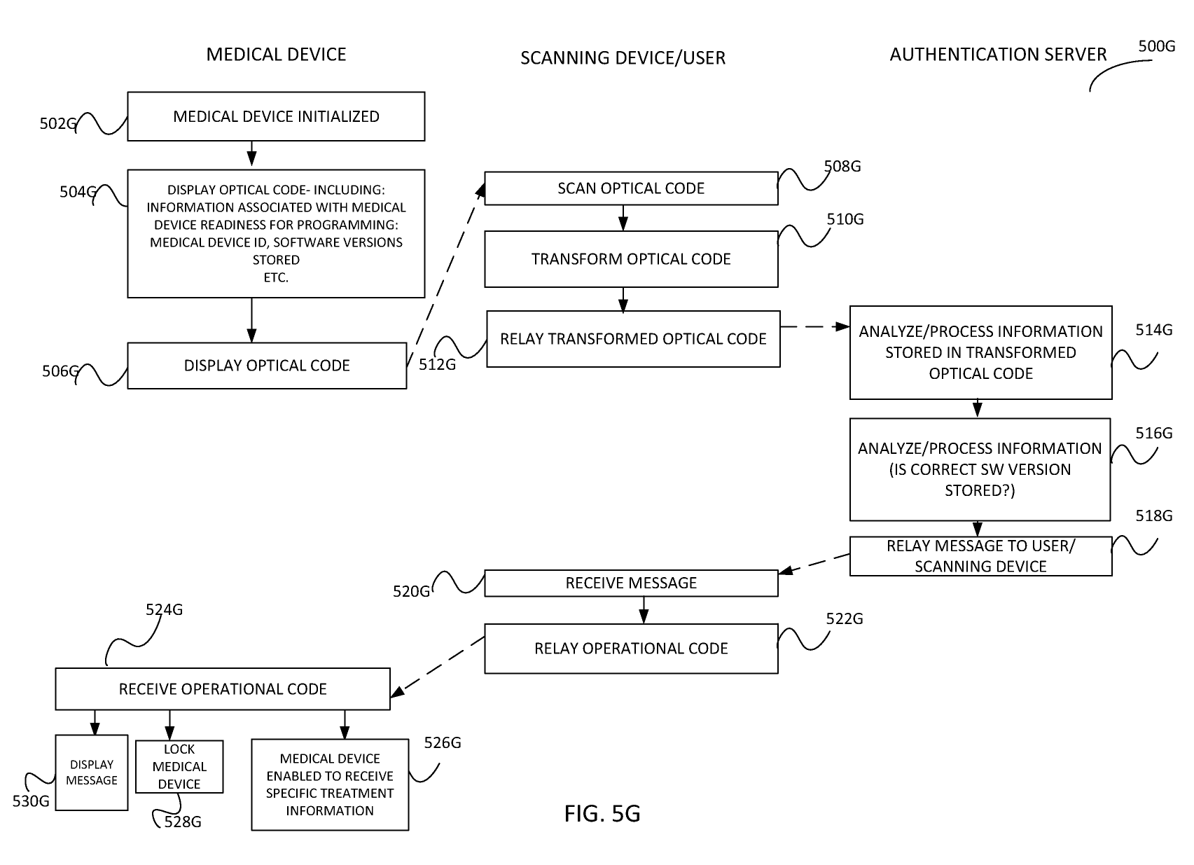

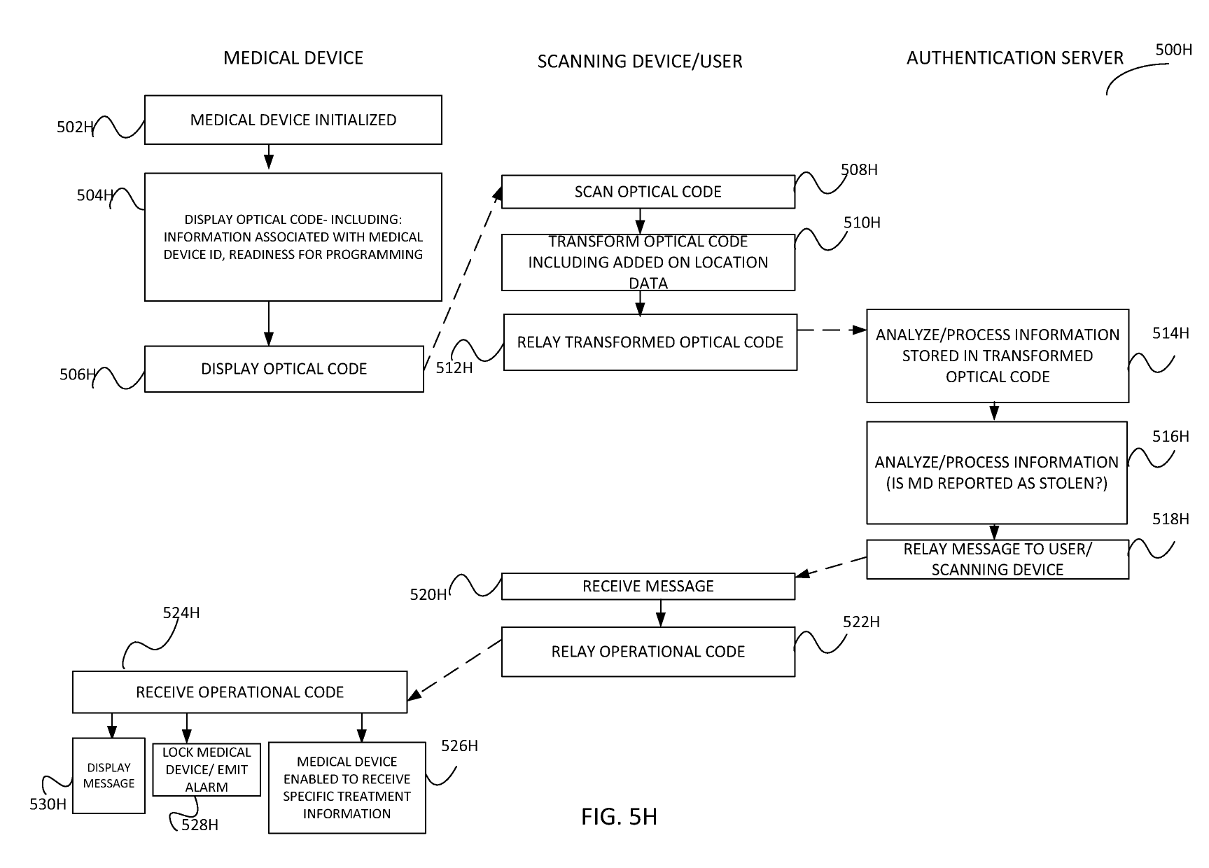

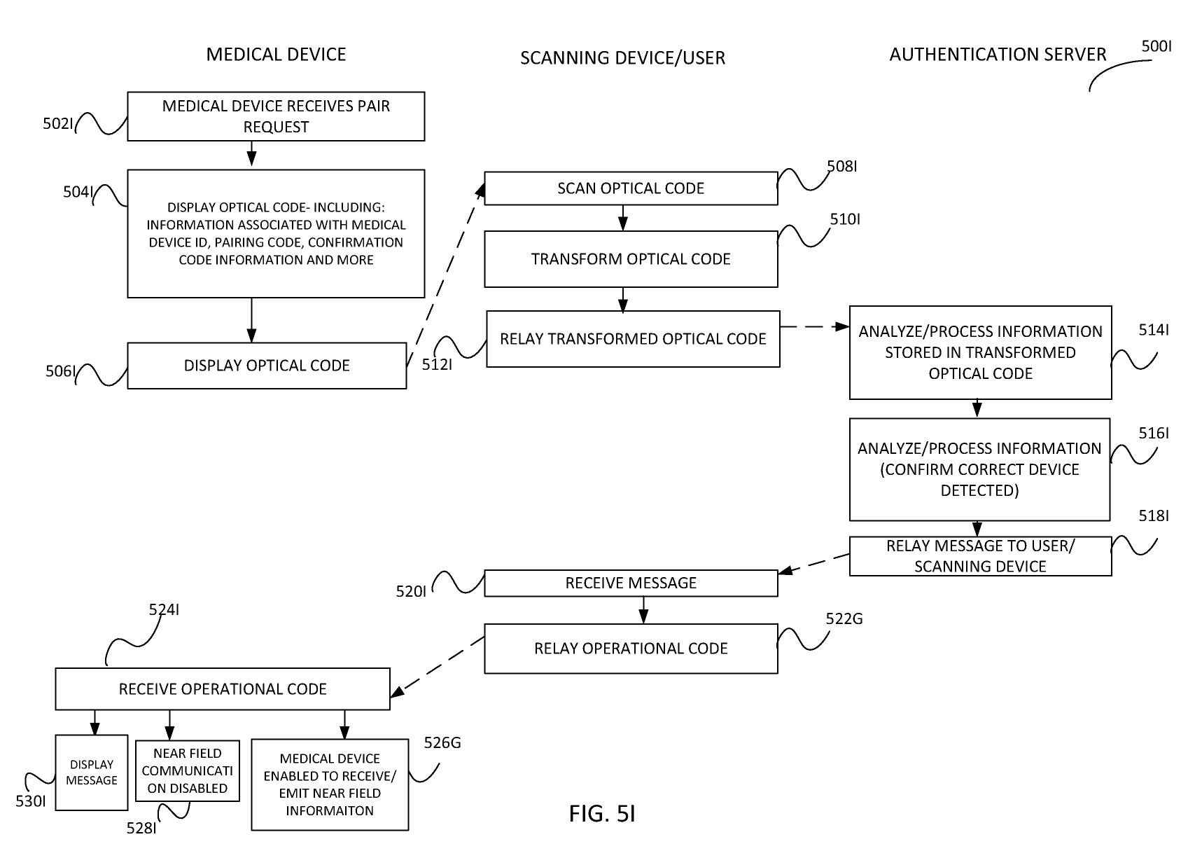

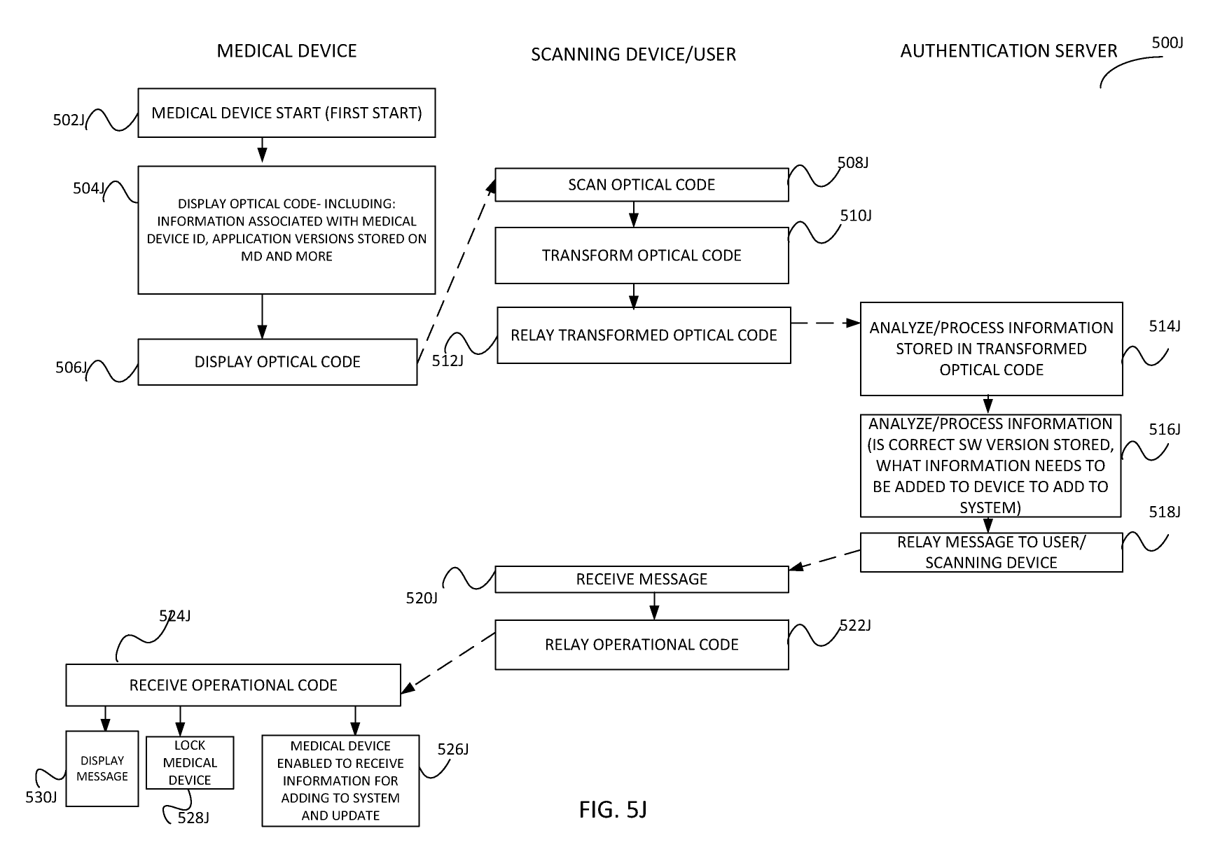

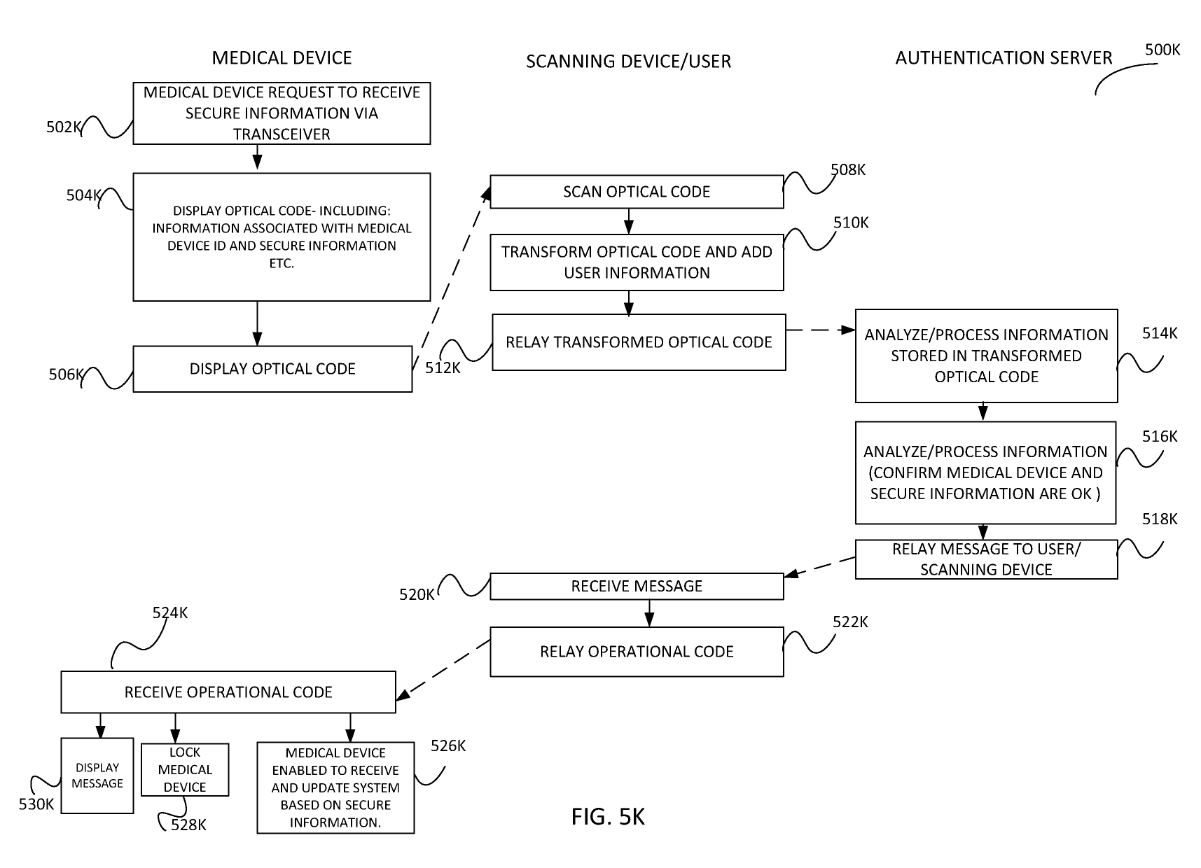

Some specific embodiments are discussed below with regard to FIGS. 5A-5K to further elaborate by way of example; additional embodiments are understood to be within the scope of the invention.

Turning to FIGS. 5A-5K shown are flowcharts (500A-500K) respectively, including the steps of an exemplary method by which medical system according to some embodiments of the present invention may perform authentication and/or management with regard to specific embodiments included within the spectrum of the invention. These examples are in no way intended to limit the scope of the invention.

In FIG. 5A, shown is an example flow which may be carried out following initialization and/or turning on of a medical device. A medical device may be turned on (step 502A) after which the medical device may calculate or determine an optical code based on information stored in the medical device (step 504A). In this example the information used and which may be encoded into the optical code or used to process additional information to be encoded into the optical code may include the medical device identification information, software version information, length of time the medical device has been used since previous calibration and more. The medical device may cause the optical code to be displayed (step 506A). A user such as a nurse may scan/obtain the optical code from the medical device using a scanning device such as a smart phone including a camera (step 508A). Optionally, the scanning device may also transform the optical code (step 510A). For example, the nurse's smart phone may add information so that the optical code is transferable via the smartphones cellular network. The scanning device may relay/transmit the transformed optical code to an M/A server (step 512A). The M/A server may receive the transformed optical code and may analyze/process the information stored in the transformed optical code as well as data stored in the M/A server and data from an associated medical device server and/or a device gateway server and determine if a version update of the software is required, does the medical device require calibration, is the medical device being looked for in the hospital network or otherwise (step 514A) and may prepare a message (step 516A) and relay the message back to the nurse's smart phone (step 518A). The nurse may receive an operational code to input into the medical device or may receive a message that the medical device requires maintenance or otherwise (step 520A). A user may relay the operational code to the medical device (step 522A). The medical device may receive the operational code into the medical device (step 524A) which may cause the medical device to: (a) transition into a state where treatment instructions may be input (step 526A) or (b) disable the medical device (step 528A) a message may be emitted on the display in response to the operational code (step 530A) which may include further instructions for example: “Please forward to medical device to a technician” or otherwise.

In FIG. 5B, shown is an example flow which may be carried out following initialization of a medical device. A medical device may conclude initialization (step 502B) after which the medical device may calculate or determine an optical code based on information stored in the medical device (step 504B). In this example the information used to be encoded into the optical code may include the medical device identification information and medical device readiness for programming such as: associated medical set loading information, battery life and more. The medical device may cause the optical code to be displayed (step 506B). A user such as an at home caregiver may scan/obtain the optical code from the medical device using a scanning device such as a smart phone including a camera (step 508B). Optionally, the scanning device may also transform the optical code (step 510B). For example, the caregivers smart phone may add information so that the optical code is transferable an email service and Wi-Fi. The scanning device may relay/transmit the transformed optical code to an M/A server (step 512B). The M/A server may receive the transformed optical code and may analyze/process the information stored in the transformed optical code as well as data stored in the M/A server and data from an associated Medical device server and/or a device gateway server and determine if the medical device is ready to proceed to receive treatment parameters or not (step 514B) and may prepare a message (step 516B) and relay the message back to the caregivers smart phone with an instant message, email, text message or otherwise (step 518B). The at home caregiver may receive an operational code to input into the medical device or may receive a message that the medical device requires modifications/corrections (step 520B). The caregiver may relay the operational code to the medical device (step 522B). The medical device may receive the operational code into the medical device (step 524B) which may cause the medical device to: (B) transition into a state where treatment parameters may be input (step 526B) or (b) disable the medical device (step 528B) a message may be emitted on the display in response to the operational code (step 530B) which may include further instructions for example: “Battery life not sufficient please charge battery” or otherwise.

In FIG. 5C, shown is an example flow which may be carried out following programming of a medical device. A medical device may identify completion of programming (step 502C) after which the medical device may calculate or determine an optical code based on information stored in the medical device (step 504C). In this example the information used to be encoded into the optical code may include the medical device identification information and medical device readiness for operation such as: user information, intended treatment program and more. The medical device may cause the optical code to be displayed (step 506C). A user such as an at home caregiver may scan/obtain the optical code from the medical device using a scanning device such as a Barcode scanner operatively connected to a handheld PDA (step 508C). Optionally, the scanning device may also transform the optical code (step 510C). For example, the caregivers PDA may add information including location associated information available on the users PDA. The scanning device may relay/transmit the transformed optical code to an M/A server (step 512C). The M/A server may receive the transformed optical code and may analyze/process the information stored in the transformed optical code as well as data stored in the M/A server and data from an associated Medical device server and/or a device gateway server and/or an HIT server and determine if the programmed treatment is compliant with the patient's EPR and/or the drug library (step 514C) and may prepare a message (step 516C) and relay the message back to the caregivers PDA via email or as a pop-up on an installed application stored on the user's PDA or otherwise (step 518C). The at home caregiver may receive an operational code to input into the medical device or may receive a message that the programmed treatment is incorrect (step 520C). The caregiver may relay the operational code to the medical device (step 522C). The medical device may receive the operational code into the medical device (step 524C) which may cause the medical device to: (a) transition into a treatment state medical device therapeutic functionality may be enabled/programmed treatment may start (step 526C) or (b) disable the medical device (step 528C) a message may be emitted on the display in response to the operational code (step 530C) which may include further instructions for example: “Programmed treatment not compliant with drug library/patient medical file/prescription” or otherwise. The above example, demonstrates how some embodiments may enable enhanced safety for example, ensuring safety protocols which call for double-checking of an input/programmed treatment even if the treatment is carried out outside of a medical healthcare environment. Some double-checking/confirmation health protocols call for a second healthcare provider such as a nurse or doctor double-check any input treatment program before administering the program to limit/decrease errors in treatment of patients.

In FIG. 5D, shown is an example flow which may be carried out following ending or completion of a therapeutic process of a medical device. A medical device may identify completion of a therapeutic process (step 502D) after which the medical device may calculate or determine an optical code based on information stored in the medical device (step 504D). In this example the information used to be encoded into the optical code may include the medical device identification information, treatment log, alarm log and more. The medical device may cause the optical code to be displayed (step 506D). A user such as a doctor may scan/obtain the optical code from the medical device using a scanning device such as a tablet or similar hand held device including a camera (step 508D). Optionally, the scanning device may also transform the optical code (step 510D). The scanning device may relay/transmit the transformed optical code to an M/A server (step 512D). The M/A server may receive the transformed optical code and may analyze/process the information stored in the transformed optical code as well as data stored in the M/A server and data from an associated medical device server and/or a device gateway server and determine if the therapeutic process was concluded successfully and if any alarms require further calibration or review of the medical device by a technician and may update an EPR of the patient with completion (or incompletion) of the treatment in a memory associated with the M/A server (step 514D). The M/A server may prepare a message (step 516D) and relay the message back to the doctor's tables for example, via Wi-Fi or otherwise (step 518D). The doctor may receive an operational code to input into the medical device (step 520D). The doctor may relay the operational code to the medical device (step 522D). The medical device may receive the operational code into the medical device (step 524D) which may cause the medical device to: (a) transition into a treatment concluded phases and be ready to receive new instructions from a user and may further store the confirmation in a memory associated with the medical device (step 526D) or (b) disable the medical device (step 528D) a message may be emitted on the display in response to the operational code (step 530D) which may include further instructions for example: “Programmed treatment concluded”, “Treatment not completed”, “medical Device requires servicing/repair” or otherwise.

In FIG. 5E, shown is an example flow which may be carried out following ending or completion of a therapeutic process of a medical device. A medical device may identify completion of a therapeutic process (step 502E) after which the medical device may calculate or determine an optical code based on information stored in the medical device (step 504E). In this example the information used to be encoded into the optical code may include the medical device identification information, treatment log, alarm log, error log and more. The medical device may cause the optical code to be displayed (step 506E). A user such as a nurse may scan/obtain the optical code from the medical device using a scanning device such as a smart phone or similar hand held device including a camera (step 508E). Optionally, the scanning device may also transform the optical code (step 510E). The scanning device may relay/transmit the transformed optical code to an M/A server (step 512E). The M/A server may receive the transformed optical code and may analyze/process the information stored in the transformed optical code as well as data stored in the M/A server and data from an associated medical device server and/or a device gateway server and determine if the therapeutic process was concluded successfully. Furthermore, the M/A server may update locally or at an associated server information associated with the alarm log and error log for statistical use and for future use (step 514E). The M/A server may prepare a message (step 516E) and relay the message back to the nurse's smart phone for example, via text message or otherwise (step 518E). The nurse may receive an operational code to input into the medical device (step 520E). The nurse may relay the operational code to the medical device (step 522E). The medical device may receive the operational code into the medical device (step 524E) which may cause the medical device to: (a) transition into a treatment concluded phases and be ready to receive new instructions from a user and may further store the confirmation in a memory associated with the medical device (step 526E) or (b) disable the medical device (step 528E) a message may be emitted on the display in response to the operational code (step 530E) which may include further instructions for example: “Programmed treatment concluded”, “Treatment not completed”, “Medical Device requires servicing/repair” or otherwise.

In FIG. 5F, shown is an example flow which may be carried out when a medical device is determined to require repair/calibration/recalibration. A medical device may identify a call to be checked by a technician or lab (step 502F) after which the medical device may calculate or determine an optical code based on information stored in the medical device (step 504F). In this example the information used to be encoded into the optical code may include the medical device identification information, software version, local parameters/definitions of a medical device system and more. The medical device may cause the optical code to be displayed (step 506F). A user such as a technician may scan/obtain the optical code from the medical device using a scanning device such as a QRcode scanner including a transceiver (step 508F). Optionally, the scanning device may also transform the optical code (step 510F). The scanning device may relay/transmit the transformed optical code to an M/A server (step 512F). The M/A server may receive the transformed optical code and may analyze/process the information stored in the transformed optical code as well as data stored in the M/A server and data from an associated medical device server and/or a device gateway server and determine if any of the stored software versions, local parameters or the like need to be removed before sending the medical device for repair (step 514F). The M/A server may prepare a message (step 516F) and relay the message back to the technician's QRcode scanner for example, via a pushed message or otherwise (step 518F). The technician may receive an operational code to input into the medical device (step 520F). The technician may relay the operational code to the medical device (step 522F). The medical device may receive the operational code into the medical device (step 524F) which may cause the medical device to: (a) transition into a generic/factory mode so that it can be sent to the lab for repair (step 526F) or (b) disable the medical device (step 528F) a message may be emitted on the display in response to the operational code (step 530F) which may include further instructions for example: “Medical device requires repair”, “Medical Device ready for servicing/repair” or otherwise. It is understood that updating the software or removing local parameters before sending a medical device to an external lab may: make sure that secure private information of a medical facility may not be shared with an external provider. Furthermore, removing local parameters may assist the lab with servicing the medical devices in a known predetermined configuration.

In FIG. 5G, shown is an example flow which may be carried out when a medical device is initialized. A medical device may identify that initialization has concluded (step 502G) after which the medical device may calculate or determine an optical code based on information stored in the medical device (step 504G). In this example the information used to be encoded into the optical code may include the medical device identification information, software version and more. The medical device may cause the optical code to be displayed (step 506G). A user such as a technician may scan/obtain the optical code from the medical device using a smart phone (step 508G). Optionally, the scanning device may also transform the optical code (step 510G). The scanning device may relay/transmit the transformed optical code to an M/A server (step 512G). The M/A server may receive the transformed optical code and may analyze/process the information stored in the transformed optical code as well as data stored in the M/A server and data from an associated Medical device server and/or a device gateway server and determine if the correct software version is stored on the medical device (step 514G). The M/A server may prepare a message (step 516G) and relay the message back to the technician's QRcode scanner for example, via WIFI or otherwise (step 518G). The technician may receive an operational code to input into the medical device (step 520G). The technician may relay the operational code to the medical device (step 522G). The medical device may receive the operational code into the medical device (step 524G) which may cause the medical device to: (a) transition into a mode wherein treatment instructions may be received(step 526G) or (b) lock the medical device (step 528G) a message may be emitted on the display in response to the operational code (step 530G) which may include further instructions for example: “Medical device prepared to receive instructions”, ““Medical Device requiring software update” or otherwise. It is understood that being able to substantially automatically check if a medical device's software is updated may simplify a robust complicated task in many medical facilities as well as ensure safety, so that a medical device may be disabled if a faulty or old software version is installed.

In FIG. 5H, shown is an example flow which may be carried out when a medical device is initialized. A medical device may identify that initialization has concluded (step 502H) after which the medical device may calculate or determine an optical code based on information stored in the medical device (step 504H). In this example the information used to be encoded into the optical code may include the medical device identification information, medical device location information and more. The medical device may cause the optical code to be displayed (step 506H). A user such as nurse may scan/obtain the optical code from the medical device using a smart phone (step 508H). Optionally, the scanning device may also transform the optical code (step 510H). The scanning device may relay/transmit the transformed optical code to an M/A server including additional location information which may be obtainable from the scanning device (step 512H). The M/A server may receive the transformed optical code and may analyze/process the information stored in the transformed optical code as well as data stored in the M/A server and data from an associated Medical device server and/or a device gateway server including a list of missing or stolen medical devices and determine if the medical device is detected as being missing or stolen (step 514H). The M/A server may prepare a message (step 516H) and relay the message back to the nurse's smart phone for example, via an email service or push message or otherwise (step 518H). The nurse may receive an operational code to input into the medical device (step 520H). The nurse may relay the operational code to the medical device (step 522H). The medical device may receive the operational code into the medical device (step 524H) which may cause the medical device to: (a) transition into a mode wherein treatment instructions may be received (step 526H) or (b) lock the medical device and (step 528H) a message may be emitted on the display in response to the operational code (step 530H) which may include further instructions for example: “Medical device prepared to receive instructions”, “Medical Device missing please update hospital employee as to location” or otherwise. It is understood that being able to identify medical devices as missing or stolen may aid in managing a fleet of medical devices in a hospital setting where many times a medical device is mislocated between the wards and/or management of a fleet of medical devices for at home use where many times patients fail to return the medical device at the end of a set of treatments.

In FIG. 5I, shown is an example flow which may be carried out when a medical device receives a pair request when a near field transmitting device requests to communicate with the medical device (send and receive information). Near field technologies enable transmittal of information from nearby devices in wireless fashion by an array of technologies such as WIFI, Bluetooth and the like. With regard to medical devices a concern with such near field technologies is that a scanning device will mistakenly converse with the wrong medical device if several medical devices are in proximity to one another. A confirmation algorithm (as understood from the application as a whole and the below example) may aid in adding required security and verification in the context of medical devices. A medical device may identify that a scanning device is emitting a pair request (step S02I) after which the medical device may calculate or determine an optical code based on information stored in the medical device (step S04I). In this example the information used to be encoded into the optical code may include the medical device identification information and more. The medical device may cause the optical code to be displayed (step S06I). A user such as a technician may scan/obtain the optical code from the medical device using a tablet including a camera and near field transmitting circuitry (step S08I). Optionally, the scanning device may also transform the optical code (step S10I). The scanning device may relay/transmit the transformed optical code to an M/A server (step S12I). The M/A server may receive the transformed optical code and may analyze/process the information stored in the transformed optical code as well as data stored in the M/A server (step S14I). The M/A server may prepare a message (step S16I) and relay the message back to the technician's tablet (step S18I). The technician may receive an operational code to input into the medical device (step S20I). The technician may relay the operational code to the medical device (step S22I). The medical device may receive the operational code into the medical device (step S24I) which may cause the medical device to: (a) transition into a mode wherein data can flow via near communication technologies between the scanning device and the medical device (step S26I) or (b) lock the medical device and (step S28I) a message may be emitted on the display in response to the operational code (step S30I) which may include further instructions for example: “Medical device prepared to receive near-field correspondence”, “Medical device not able to receive near filed transmissions” or otherwise.

In FIG. 5J, shown is an example flow which may be carried out when a user wants to add a medical device to a fleet of medical devices in a medical facility or for home use. A fleet of medical devices may be characterized by specific software versions that preferably are aligned and possibly local parameters/definitions. A medical device may be in a booted mode, for example just received at a medical facility (but of the box′) or may receive an input that it is requested to join a fleet (step 502J) after which the medical device may calculate or determine an optical code based on information stored in the medical device (step 504J). In this example the information used to be encoded into the optical code may include the medical device identification information, application versions stored on MD, any history logs and more. The medical device may cause the optical code to be displayed (step 506J). A user such as a technician may scan/obtain the optical code from the medical device using a tablet including a camera (step 508J). Optionally, the scanning device may also transform the optical code (step 510J). The scanning device may relay/transmit the transformed optical code to an M/A server (step 512J). The M/A server may receive the transformed optical code and may analyze/process the information stored in the transformed optical code as well as data stored in the M/A server (step 514J). The M/A server may prepare a message (step 516J) and relay the message back to the technician's tablet (step 518J). The technician may receive an operational code to input into the medical device (step 520J). The technician may relay the operational code to the medical device (step 522J). The medical device may receive the operational code into the medical device (step 524J) which may cause the medical device to: (a) transition into a mode wherein the medical device is open to version updates and local parameter definitions including which versions are expected and more (step 526J) or (b) lock the medical device and (step 528J) a message may be emitted on the display in response to the operational code (step 530J) which may include further instructions for example: “Medical device prepared to connect to fleet”, “Medical device not able to connect to fleet” or otherwise.

In FIG. 5K, shown is an example flow which may be carried out when a medical device either expects a wireless transmission to be received or actually receives a connection request. A wireless transmission may enable remote update of information to the machine, however, in the context of medical devices additional confirmation may be required for regulatory and safety reasons. A confirmation algorithm (as understood from the application as a whole and the below example) may aid in adding required security and verification to allow remote update of the medical device and associated parameters with local confirmation and verification involving a user. A medical device may identify that a remote device is emitting a wireless transmission or that a wireless transmission is expected (step 502K) after which the medical device may calculate or determine an optical code based on information stored in the medical device (step 504K). In this example the information used to be encoded into the optical code may include the medical device identification information and more. The medical device may cause the optical code to be displayed (step 506K). A user such as a technician may scan/obtain the optical code from the medical device using a smart phone including a camera (step 508K). Optionally, the scanning device may also transform the optical code (step 510K). The scanning device may relay/transmit the transformed optical code to an M/A server (step 512K). The M/A server may receive the transformed optical code and may analyze/process the information stored in the transformed optical code as well as data stored in the M/A server to confirm/verify that the medical device identified by the user (by scanning the optical code) is the expected medical device (step 514K). The M/A server may prepare a message (step 516K) and relay the message back to the technician's smart phone (step 518K). The technician may receive an operational code to input into the medical device (step 520K). The technician may relay the operational code to the medical device (step 522K). The medical device may receive the operational code (step 524K) which may cause the medical device to: (a) transition into a mode wherein data can flow between the medical device and the remote device (step 526K) or (b) lock the medical device and (step 528K) a message may be emitted on the display in response to the operational code (step 530K) which may include further instructions for example: “Medical device prepared to receive remote correspondence”, “Medical device not able to receive near field transmissions” or otherwise.

While certain features of the invention have been illustrated and described herein, many modifications, substitutions, changes, and equivalents will now occur to those skilled in the art. It is, therefore, to be understood that the appended claims are intended to cover all such modifications and changes as fall within the true spirit of the invention.

What is claimed:

1. A medical device comprising:
a display;
a therapeutic component to provide therapeutic functionality; and
a controller to regulate operation of said therapeutic component and including processing logic to: (a) generate and render on said display an optical symbol indicative of an intended operational state of said therapeutic component; (b) receive from a user an operation code, and (c) responsive to validation of the operation code to enable the intended operational state.

2. The medical device of claim 1 further comprising a memory accessible by said controller, said memory configured to store medical device information.

3. The medical device of claim 2 wherein said medical device information includes at least one of the group consisting of: therapeutic component operational log, medical device identification, software version information and medical device history.

4. The medical device of claim 2, wherein said optical symbol is further indicative of medical device information retrievable from said memory.

5. The medical device of claim 1 wherein said intended operational state is at least one of the states selected from the group consisting of: initialization, receive therapeutic operation parameters, begin therapeutic operation, end therapeutic information, log new medical device onto system, hold therapeutic functionality due to unusual event during operation and prepare medical device for repair.

6. The medical device of claim 1, wherein said therapeutic component is a fluid therapy pump.

7. The medical device of claim 1, further comprising a transceiver, wherein said intended operational state is indicative of a requested communication connection between said transceiver and a remote server, and the intended operational state is an open communication between said transmitter and the remote server so that said processing logic is configured to receive secure information from said remote server through said transceiver.

8. A medical system comprising:
a medical device including a controller to generate and render on a display an optical symbol indicative of an intended operational state of said therapeutic component; and
a first server to receive a processed optical symbol through a hybrid communication path, and to produce an operation code at least partially based on said processed optical symbol;
wherein said medical device is further configured to receive said operation code and responsive to validation of the operation code is configured to enable the intended operation state.

9. The system of claim 8, wherein said first server is an authentication server.

10. The system of claim 8, wherein said first server is a management server.

11. The system of claim 8, wherein said medical device further comprises a display and a therapeutic component to provide therapeutic functionality.

12. The medical device of claim 11, wherein said controller is further configured to regulate operation of said therapeutic component.

13. The system of claim 8, further comprising a medical device memory accessible by said controller to store medical device information and wherein said optical symbol is further indicative at least a part of said medical device information stored in said memory.

14. The system of claim 8, wherein said first server includes a server memory configured to store medical device information.

15. The system according to claim 14, wherein said first server is further configured to utilize both said processed optical symbol and said medical device information to produce said processed optical code.

16. The system according to claim 8, wherein said medical device further comprises a transceiver, wherein said intended operational state is indicative of a requested communication connection between said transceiver and a remote server, and the intended operational state is an open communication between said transmitter and the remote server so that said processing logic is configured to receive secure information from said remote server through said transceiver.

17. The system according to claim 8, wherein said remote server is said first server.

18. The system according to claim 8, wherein said medical device further comprises a near field transceiver, wherein said intended operational state is indicative of a requested near field communication connection between said medical device and a proximate scanning device included in the hybrid communication path, and the intended operational state is an open communication between said medical device and the scanning device.

19. A medical device comprising:
a display;
a diagnostic component to provide diagnostic functionality; and
a controller to regulate operation of said diagnostic component and including processing logic to: (a) generate and render on said display an optical symbol indicative of a first state of said medical device; (b) receive from a user an operation code, and (c) responsive to validation of the operation code to enable transition to a second state of said medical device.
